(19) United States
(12) Patent Application Publication
(10) Pub. No.: US 2025/0072816 A1
(43) Pub. Date: Mar. 6, 2025

(54) FRUSTRATED TOTAL INTERNAL REFLECTION (FTIR)-BASED HEALTH PARAMETER DETECTION SYSTEMS, METHODS, AND DEVICES

(71) Applicant: GS-HealthMatrix, LLC, Los Angeles, CA (US)

(72) Inventors: Joseph Schwab, Los Angeles, CA (US); Amirhossein Yazdkhasti, Los Angeles, CA (US); Hamid Ghaednia, Los Angeles, CA (US)

(73) Assignee: GS-HealthMatrix, LLC, Los Angeles, CA (US)

(21) Appl. No.: 18/819,309

(22) Filed: Aug. 29, 2024

Related U.S. Application Data
(60) Provisional application No. 63/579,605, filed on Aug. 30, 2023.
(60) Provisional application No. 63/579,616, filed on Aug. 30, 2023.
(60) Provisional application No. 63/579,627, filed on Aug. 30, 2023.
(60) Provisional application No. 63/579,633, filed on Aug. 30, 2023.
(60) Provisional application No. 63/579,640, filed on Aug. 30, 2023.
(60) Provisional application No. 63/579,647, filed on Aug. 30, 2023.
(60) Provisional application No. 63/579,663, filed on Aug. 30, 2023.

(51) Int. Cl. A61B 5/00; A61B 90/00

(52) U.S. Cl. CPC A61B 5/4023; A61B 5/0064; A61B 5/0079; A61B 5/6895; A61B 5/742; A61B 90/06; A61B 2090/064; A61B 2560/0233; A61B 2560/0431; A61B 2560/0462; A61B 2562/046

(57) ABSTRACT

Systems, methods, and devices include a health parameter detection device. The device includes a platform with a measurement surface being a first surface. The measurement surface is operable to contact a target area of a user. The device includes one or more light sources disposed adjacent to the transparent material and operable to transmit light into the transparent material. Also, the device includes an interior mirror forming a first angle with the measurement surface; and/or a camera, such that the camera is operable to receive a scattered light caused by a frustrated total internal of reflection (FTIR) event occurring at the measurement surface and reflecting from the mirror. The platform can form part of at least one of a treadmill machine, an elliptical machine, a rowing machine, a stair stepping machine, or a leg press machine.

### CROSS-REFERENCE TO RELATED APPLICATIONS

This application claims priority to U.S. Provisional Application Ser. No. 63/579,605 filed Aug. 30, 2023 and titled “FRUSTRATED TOTAL INTERNAL REFLECTION (FTIR) SURFACE TOPOGRAPHY AND COMPOSITION ANALYSIS SYSTEMS, METHODS, AND DEVICES;” U.S. Provisional Application Ser. No. 63/579,616 filed Aug. 30, 2023 and titled “SYSTEMS, METHODS, AND DEVICES OF WEARABLE ELECTRO-ACOUSTIC MONITORING;” U.S. Provisional Application Ser. No. 63/579,627 filed Aug. 30, 2023 and titled “SYSTEMS, METHODS, AND DEVICES FOR ACOUSTICALLY ENHANCING IMPLANTS;” U.S. Provisional Application Ser. No. 63/579,633 filed Aug. 30, 2023 and titled SYSTEMS, METHODS, AND DEVICES WITH SENSORS HAVING MULTIPLE DETECTION SIGNAL TYPES;” U.S. Provisional Application Ser. No. 63/579,640 filed Aug. 30, 2023 and titled MULTI-DEVICE HEALTH PARAMETER MONITORING SYSTEMS, METHODS, AND DEVICES;” U.S. Provisional Application Ser. No. 63/579,647 filed Aug. 30, 2023 and titled FRUSTRATED TOTAL INTERNAL REFLECTION (FTIR)-BASED HEALTH PARAMETER DETECTION SYSTEMS, METHODS, AND DEVICES;” and U.S. Provisional Application Ser. No. 63/579,663 filed Aug. 30, 2023 and titled “SYSTEMS, METHODS, AND DEVICES FOR NEUROLOGICAL AND/OR MUSCOSKELETAL PARAMETER CHARACTERIZATION;” the entireties of which are herein incorporated by reference.

### BACKGROUND

Conventional devices using optical analysis for health characterizations typically assume a constant mechanical property around the sample area without considering any spatial differences or surface characteristics of the sample area. These systems typically use only a single wavelength of visible light (e.g., red light). Furthermore, conventional optical analysis devices are very large, heavy, and are not easy to move between exam rooms. As such, the health characterizations performed with conventional optical analysis devices are limited.

It is with these observations in mind, among others, that various aspects of the present disclosure were conceived and developed.

### SUMMARY

Systems, methods, and devices disclosed herein can address the aforementioned issues. For instance, a health parameter detection device can include a body with a platform formed of a transparent material. The platform can include a measurement surface forming a first surface of the body, and the measurement surface can be operable to contact a target area of a user. The device can also include one or more light sources disposed adjacent to the transparent material and operable to transmit light into the transparent material. Also, the device can include a mirror disposed at an interior of the body and forming a first angle with the measurement surface; and/or a camera disposed at least partially in the body, such that the camera is operable to receive a scattered light caused by a frustrated total internal of reflection (FTIR) event occurring at the measurement surface and reflecting from the mirror.

In some examples, the device includes one or more wheels extending from a second surface of the body. The device can also include a handle formed into a third surface of the body. Moreover, the device can include a mounting frame disposed between the platform and the mirror, substantially parallel to the platform; and/or one or more force sensors, disposed between the platform and the mounting frame, which can be operable to detect a force applied to the measurement surface. Additionally, the one or more light sources can include a row of light emitting diodes (LEDs) disposed along a side of the transparent material. Also, the mirror can include a first mirror; and/or the device can further include a second mirror in the interior of the body forming a third angle with the measurement surface such that the second mirror receives light reflected from the first mirror. Furthermore, the device can include a third mirror in the interior of the body forming a fourth angle with the measurement surface such that the third mirror reflects light, reflected from the second mirror and received at the third mirror, towards the camera. The third mirror can be a curved mirror, and/or the device can also include a lens positioned between the third mirror and the camera. The one or more light sources can include a plurality of different frequency LEDs; and/or the scattered light can be caused by a plurality of FTIR events resulting from powering the plurality of different frequency LEDs, one frequency at a time, such that the health parameter detection device constructs a nano scale or micro scale 3D topology from the plurality of FTIR events.

In some scenarios, a health parameter detection system can include a platform formed of a transparent material. The platform can include a measurement surface operable to contact a target area of a user. Also, the multi-sensor scanning system can include a light source operable to transmit light into the transparent material; one or more camera directed at the transparent material such that the one or more camera is operable to receive a scattered light caused by a frustrated total internal reflection (FTIR) event occurring at the measurement surface because of contact with the target area; and/or one or more motors operable to move the measurement surface.

In some examples, the system also includes a computer-readable memory device storing instructions that, when executed by one or more processor, can cause the health parameter detection system to collect calibration light data by powering the light source for a predetermined calibration time interval, the light source including a calibration set of LEDs; collect test data by performing a two-part assessment of a subject; normalize the test data using the calibration light data, anthropometric data, or patient demographic, to generate normalized test data; and/or cause a visual representation of the normalized test data to be presented at a graphical user interface on a display of a clinician computing device. Additionally, the instructions, when executed by the one or more processor, can further cause the health parameter detection system to generate a skin surface calibration profile indicating one or more optical properties of a bottom surface of a foot. The one or more camera can include a plurality of cameras forming an array directed at an underside of the platform. Also, the platform can include at least one of a flexible running belt including the transparent material for receiving the light from the light source; and/or a rigid deck around which the flexible running belt wraps. The rigid deck can include the transparent material for receiving the light from the light source. Additionally, the platform can include a plurality of parallel, rigid planks extending from a first side of the platform to a second side of the platform. The platform can form part of at least one of a treadmill machine; an elliptical machine; a rowing machine; a stair stepping machine; and/or a leg press machine.

In some instances, a method of health parameter detection includes collecting calibration light data by powering a light source for a predetermined calibration time interval, the light source being adjacent to a transparent material forming a measurement surface; collecting test data by powering the light source during a testing time interval to perform a two-part assessment of a subject, the test data corresponding to one or more frustrated total internal reflection (FTIR) events occurring at the measurement surface caused by powering the light source for a testing time interval; normalizing the test data to generate normalized test data; and/or causing a visual representation of the normalized test data to be presented at a graphical user interface on a display of a clinician computing device.

In some scenarios, the light source can include a calibration set of light emitting diodes (LEDs) including LEDs of different frequencies corresponding to a contact surface of a calibration subject. Also, the two-part assessment can include generating first position data corresponding to a position of the subject; and/or generating second position data corresponding to the position with vestibular inhibition. Moreover, the test data can include a balance distribution generated by calculating a difference between the first position data and the second position data.

### BRIEF DESCRIPTION OF THE DRAWINGS

The foregoing summary, as well as the following detailed description, will be better understood when read in conjunction with the appended drawings. For the purpose of illustration, there is shown in the drawings certain embodiments of the disclosed subject matter. It should be understood, however, that the disclosed subject matter is not limited to the precise embodiments and features shown. The accompanying drawings, which are incorporated in and constitute a part of this specification, illustrate an implementation of systems and methods consistent with the disclosed subject matter and, together with the description, serves to explain advantages and principles consistent with the disclosed subject matter, in which:

FIG. 1 illustrates an example health parameter detection system using a platform to generate Frustrated Total Internal Reflection (FTIR) events.

FIG. 2 illustrates an example health parameter detection system using a platform with a compact form factor to generate FTIR events.

FIG. 3 illustrates an example health parameter detection system including various components of a platform with a compact form factor to generate FTIR events.

FIGS. 4A and 4B illustrate an example health parameter detection system using a moving platform comprising a flexible running belt to generate FTIR events.

FIG. 5 illustrates an example health parameter detection system using a moving platform comprising a flexible running belt around a rigid deck to generate FTIR events.

FIG. 6 illustrates an example health parameter detection system using a moving platform comprising a plurality of planks to generate FTIR events.

FIG. 7 illustrates an example health parameter detection system using a platform of an exercise machine to generate FTIR events.

FIG. 8 illustrates an example method of health parameter detection, which can be performed by any of the systems depicted in FIGS. 1-7.

FIG. 9 illustrates an example method of health parameter detection, which can be performed by any of the systems depicted in FIGS. 1-7.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the presently disclosed technology or the appended claims. Further, it should be understood that any one of the features of the presently disclosed technology may be used separately or in combination with other features. Other systems, methods, features, and advantages of the presently disclosed technology will be, or become, apparent to one with skill in the art upon examination of the figures and the detailed description. It is intended that all such additional systems, methods, features, and advantages be included within this description, be within the scope of the presently disclosed technology, and be protected by the accompanying claims.

Further, as the presently disclosed technology is susceptible to embodiments of many different forms, it is intended that the present disclosure be considered as an example of the principles of the presently disclosed technology and not intended to limit the presently disclosed technology to the specific embodiments shown and described. Any one of the features of the presently disclosed technology may be used separately or in combination with any other feature. References to the terms “embodiment,” “example,” and/or the like in the description mean that the feature and/or features being referred to are included in, at least, one aspect of the description. Separate references to the terms “embodiments,” “examples,” and/or the like in the description do not necessarily refer to the same embodiment and are also not mutually exclusive unless so stated and/or except as will be readily apparent to those skilled in the art from the description. For instance, a feature, structure, process, step, action, or the like described in one embodiment may also be included in other embodiments, but is not necessarily included. Thus, the presently disclosed technology may include a variety of combinations and/or integrations of the examples described herein. Additionally, all aspects of the present disclosure, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

Any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described. The term “real-time” or “real time” means substantially instantaneously.

Lastly, the terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B and C.” An exception to this definition will occur only when a combination of elements, functions, steps or acts are in some way inherently mutually exclusive.

Systems, methods, and device include an improved health parameter characterization device that can perform pressure mapping based on the occurrence of frustrated total internal reflection (FTIR) events. The device can include one or more cameras to capture light intensity values generated at a transparent medium, which are used to measure a pressure distribution of a subject or object contacting the transparent medium. These systems, methods, and devices can use the transparent medium to measure pressure caused by, and surface characteristics of, human tissue at the contact surface. The techniques disclosed herein can also be used in several other fields of study, such as contact mechanic, electrical contacts, rolling element bearings, sealing mechanics (e.g., subsea), shields, and so forth.

In some instances, a health parameter characterization device can use different wavelengths of light inside a transparent medium. This can result in a boundary condition at which an electromagnetic field prevents photons from escaping the transparent medium until an external object disturbs this electromagnetic wave. When an object (e.g., a surface of a body part) interferes with this boundary condition or, in other words, contacts the transparent medium, the transparent medium emits the photons. The more pressure between the object and the surface, the closer the surface features of the object get to the transparent medium, and the more photons will scatter. Therefore, a camera recording the images of the contacting areas can be used to measure the light intensities at different locations, and the light intensities can be transformed into a pressure map for display at one or more computing devices. Moreover, simultaneously with or alternatively to generating the pressure map, the techniques disclosed herein can generate one or more of a perfusion map, a heat map, an electrical activity map, and/or other types of health parameter characterizations based on the contact surface interface with the body part.

As discussed herein, electromagnetic waves can refer to wavelengths of light smaller and larger than visible light as well as the visible light (e.g., ultraviolet, visible light and/or near, mid and long range infrared), and the transparent medium can refer to a medium that does not shield the electromagnetic waves at the wavelengths that are used in the specific application. For example, if transparent medium is used for infrared light, it does not mean that the medium is see-though but rather that it does not shield the infrared electromagnetic waves. In other words, the “transparency” of the transparent medium can be particularized for a specific frequency.

In some scenarios, the health parameter characterization device can be a standing platform with a reduced height (e.g., below 14 inches in height), which can reduce safety risks, especially for patients that already suffer from imbalance, as compared to taller measurement devices. The system can include at least two configurations of components, which can use one or more 3D curved mirrors and can form a reduced height profile for the device. In other examples, the configuration of components can include a plurality of cameras used to reduce the height of the device.

Furthermore, a variety of exercise machines and/or rehabilitation devices can be made with the technology disclosed herein. For instance, the transparent medium can be formed into one or more of a treadmill machine, an elliptical machine, a rowing machine, a stair stepping machine, or a leg press machine, which can provide an improvement to evaluations during workouts or physical therapy. Also, these highly sophisticated, multi-functional pressure plates can be incorporated into Virtual Reality (VR) environments.

The systems, methods, and devices can include a health parameter characterization procedure used with patients being tested. The technology disclosed herein can have many different application which each can require a unique process before, after, and during the testing procedure. An example health parameter characterization procedure with an application to improve a balance assessment is also disclosed herein.

Additional advantages and benefits will become apparent from the detailed description below.

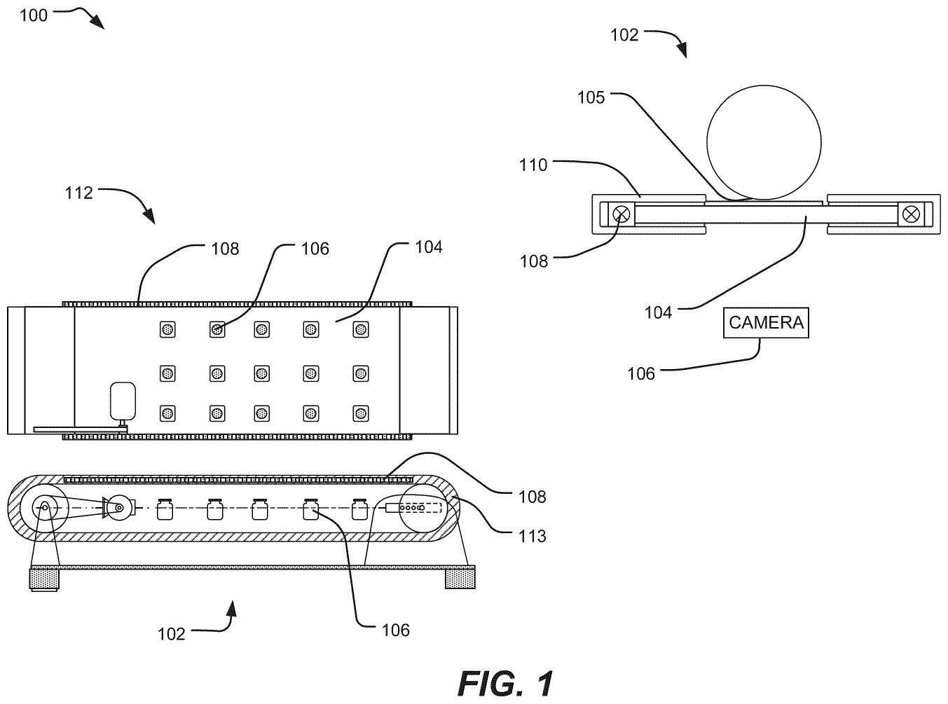

FIG. 1 depicts an example system 100 for performing a health parameter detection and/or characterization. The system 100 can include a health parameter detection device 102 which generates a pressure map, a perfusion map, a surface topology, and/or a material composition by analyzing a surface of a body part (e.g., a sample surface 105) pressed against a transparent medium 104. The surface of the body part can be a bottom of foot of a person standing or pushing against the transparent medium 104. The health parameter detection device 102 can use the concept of FTIR to trap different wavelengths of electromagnetic waves inside the transparent medium 104 (e.g., a glass sheet, a plastic sheet, or so forth). In this condition, if any surface feature of the body part (e.g., the sample surface 105) gets closer to the transparent medium 104 than the wavelength of the trapped light, photons start scattering from the contacting areas. Therefore, one or more cameras 106 and/or a microscope under the glass medium can record those photons. In some instances, different wavelengths of electromagnetic waves from ultraviolet range to visible and infrared waves are generated by one or more LEDs (e.g., a plurality of LEDs 108). The plurality of LEDs 108 can include a wide range of LEDs, for instance, between a 10 nm wavelength and a 4000 nm wavelength.

One or more cameras 106 on the other side of the transparent medium 104, opposite from the measurement surface, can record the scattered photons. The amount of photons and/or an amplitude of photons at a particular area of the contact area can correspond to a pressure applied to the transparent medium 104, such that a pressure map can be generated from this data. Also, the LEDs with different wavelengths can be turned on one frequency at a time (e.g., in a one-by-one sequence), for instance, from smaller wavelengths to larger wavelengths. The one or more cameras 106 can record the scattered photos at the different LED illumination stage to record slices of the contacting surface at different distances to the glass surface. Moreover, a particular set of frequencies can be illuminated at one time, corresponding to a particular use case. The different distances from the glass surface correspond to the different wavelengths of the LEDs. By combining these pictures, a 3D nanoscale or microscale surface topography of the contact area (e.g., the body part pressed against the transparent medium 104) can be generated. In other words, the health parameter detection device 102 can be used to generate images of different distance slices of the object against the glass medium. The health parameter detection device 102 can operate as a nano scale 3D scanner which constructs the 3D profile of the contacting area. From these surface topographies, a friction factor, a roughness factor, and/or other mechanical or optical characteristics of the contact surface can be determined.

Moreover, by analyzing the amplitude of the receiving wave for each of these wavelengths, the health parameter detection device 102 can simultaneously determine the material composition of the contacting body part (e.g., a water content, a saline percent, an oxygen content, etc.). Wavelengths of light outside the visible range, especially in near infrared range from wavelengths 800 nm to 4000 nm, can be used. FTIR spectroscopy for determining the material can be performed simultaneously with generating the pressure map, the perfusion map, and/or the 3D surface topology. One or more cameras 106 (e.g., additionally or alternatively to light spectrometers) can detect electromagnetic waves scattered at different frequencies one at a time, or in a particular frequency set, and only at the wavelengths needed for the material composition analysis. For example, if the health parameter detection device 102 is used to check the oxygen level at the contact surface, LEDs that emit certain wavelengths of EM waves that can help detect oxygen are used (e.g., between 1400-1600 nm). In some examples, the chemical composition analysis performed with a visible light camera 106 and multiple different wavelengths is an improvement over other FTIR spectroscopy devices that may use light spectrometers.

In some examples, a pressure distribution can be determined additionally or alternatively to a perfusion distribution. This can be especially useful to patients with diabetes or neuropathy, for which a perfusion distribution under the foot can be determined by measuring changes in the pressure map and/or the surface topologies over time and correlating those changes to known blood flow regions (e.g., using one more machine-learning techniques).

Moreover, the health parameter detection device 102 can include one or more interchangeable LED assemblies that correspond to a particular use case, a particular component of the material composition, and so forth. The glass medium can also have an electrically conductive portion, such as transparent, electrically conducting ink, formed onto the contact surface of transparent medium 104. The electrically conducting ink can be used to send and/or receive electrical signals into the subject via the contact surface. Furthermore, other portions of the transparent medium 104 (e.g., outside the measurement surface portion) can be covered with a shield 110 or an opaque material to trap the photons in transparent medium 104.

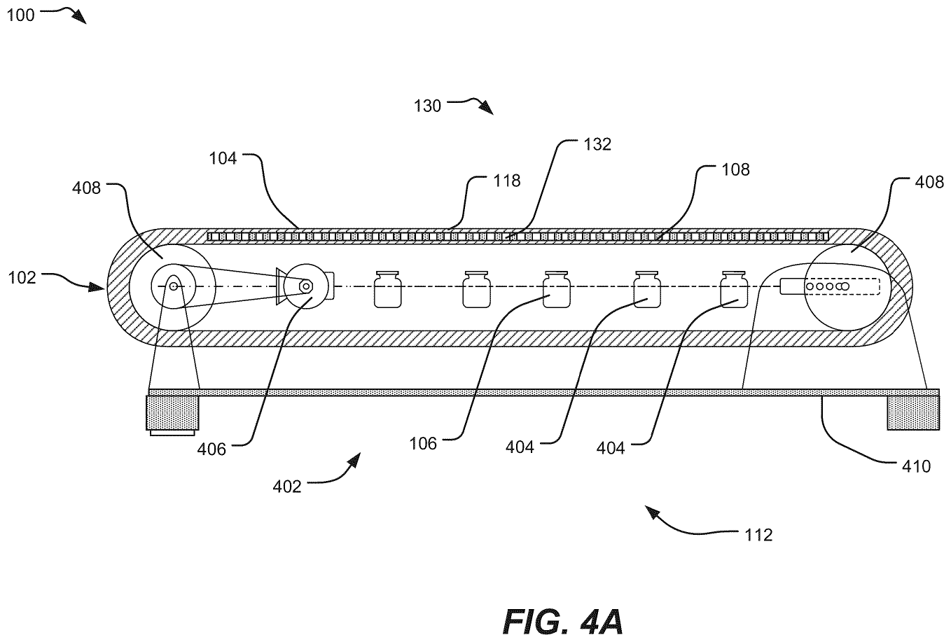

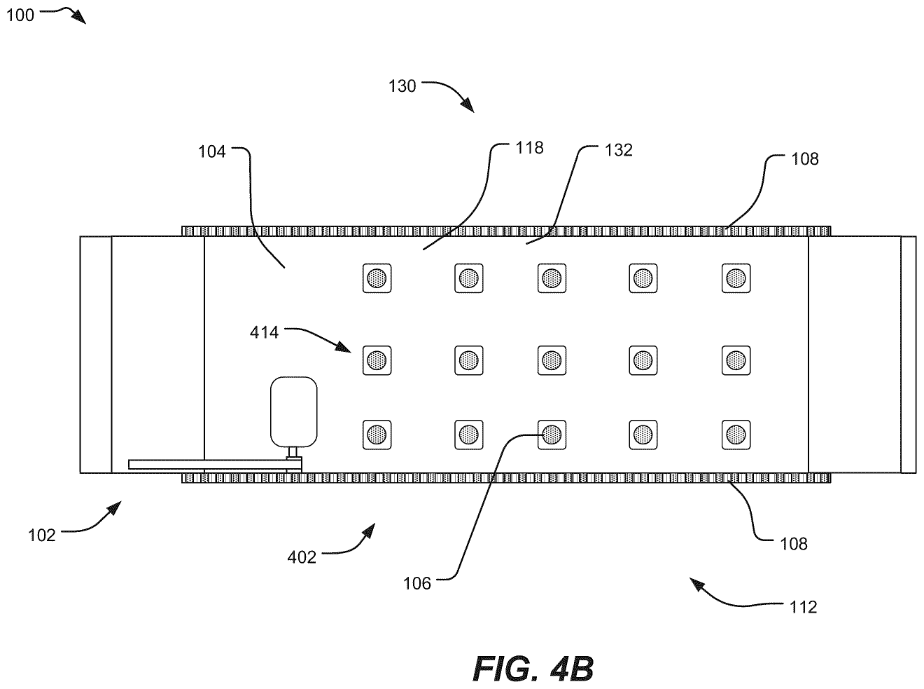

In some scenarios, the transparent medium 104 can be formed into a top portion of a locomotion device 112, such as an exercise device or a physical therapy device for humans and/or animals. For example, the transparent medium 104 can be formed into a flexible running belt 113, rigid deck, a plurality of planks, or combinations thereof, as discussed in greater detail below regarding FIGS. 4A-7.

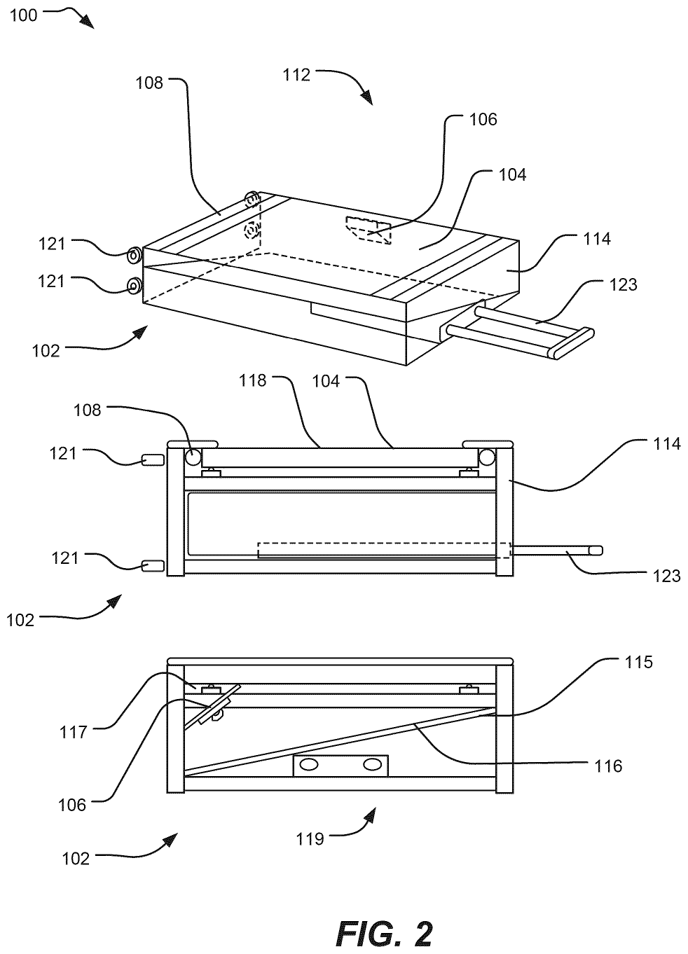

FIG. 2 depicts an example system 100 including the health parameter detection device 102. The system 100 depicted in FIG. 2 can form at least a portion of the system 100 depicted in FIG. 1. The health parameter detection device 102 can include a body 114 with one or more mirrors disposed in an interior of the body 114. For instance, the body 114 can have the transparent medium 104 defining a first surface (e.g., a top surface) of the body 114, and a first mirror can be an inclined mirror 116 disposed below the transparent medium 104. The inclined mirror 116 can be positioned opposite the top measurement surface 118 of the transparent medium 104, which can be exposed. The inclined mirror 116 can form an angle with the body 114 (e.g., the first surface) such that scattered light incident on the inclined mirror 116 from the transparent medium 104 is reflected towards the one or more cameras 106. The one or more cameras 106 can be positioned in an upper corner 117 of the interior of the body 114 facing downwards at an angle towards the inclined mirror 116.

In some instances, this inclined mirror-camera system 119 can decrease the overall height of the health parameter detection device 102 when the health parameter detection device 102 is in measurement-taking position. The one or more cameras 106 can be placed beneath the glass top (e.g., the transparent medium 104) and can look down at the inclined mirror 116. Moreover, to provide portability and mobility, two, three, or four wheels 121 can extend from one side of the body 114, and an extendable and/or adjustable handle 123 can extend from another side of the body 114. As such, the health parameter detection device 102 can be transitionable between a measurement-taking position and a transport position, which can include a 90° rotational difference from the measurement-taking position.

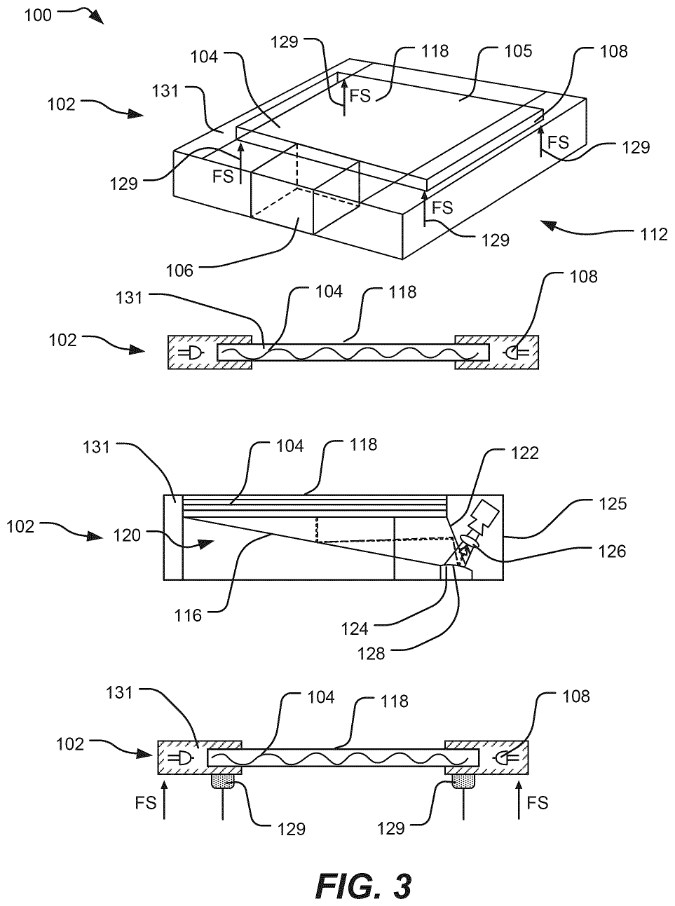

FIG. 3 depicts an example system 100 including the health parameter detection device 102 with multiple mirrors 120. The system 100 depicted in FIG. 3 can form at least a portion of the system 100 depicted in FIG. 1.

For examples, the health parameter detection device 102 can include a second mirror 122 that forms another angle with the first surface (e.g., the top measurement surface 118) of the body 114. As such, the second mirror 122 can receive light reflected from the inclined mirror 116, and can reflect the light towards a third mirror 124. The third mirror 124 can, in turn, reflect the light from the second mirror 122 towards the one or more cameras 106. The first mirror 115, the second mirror 122, and/or the third mirror 124 can comprise a 3D curved mirror (e.g., instead of an inclined mirror), and one or more lenses 126 can be disposed between the first mirror 115, the second mirror 122, and/or the third mirror 124, which can further decrease a height dimension 125 of the health parameter detection device 102. Accordingly, these configurations can improve the portability of the health parameter detection device 102 such that the device can be designed to be comparable in size to at-home scales. Accordingly, the health parameter detection device 102 can be much simpler to move, safer, and lighter. Furthermore, the camera(s) 106 and the curved mirror system could be designed as a modular part that could be replaceable or modifiable. In these examples, the camera(s) 106 can be placed beneath the glass top (e.g., the transparent medium 104) and can look away from the glass and into the curved mirror 128. Furthermore, the health parameter detection device 102 can include one or more force sensors 129 disposed below the transparent medium 104 for measuring a force applied to the top measurement surface 118.

In some examples, a configuration of the health parameter detection device 102 can include multiple cameras, which can increase a field of vision of the detection system and/or can further reduce the height dimension of the health parameter detection device 102. As noted above, the camera(s) 106 can include any combination of visible light cameras, infrared cameras, UV cameras, and so forth. In addition to decreasing the height of the health parameter detection device 102, a plurality of cameras 106 can be used for creating large and/or elongated platforms 131 that measure pressure distributions, perfusion distributions, electrical distributions, surface topologies, and so forth. These platforms 131 can be integrated into one or more locomotion device 112, as discussed in greater detail below regarding FIGS. 4-6.

FIGS. 4-6 depict example locomotion devices 112 that include the components of the health parameter detection device 102. These locomotion device 112 can be integrated with the health parameter detection device 102 to provide multiple mapping capabilities based on the various types of collected data. The systems 100 depicted in FIGS. 4-6 can form at least a portion of the system 100 depicted in FIG. 1.

The locomotion device(s) 112 disclosed herein can address some of the obstacles of using pressure mapping devices, especially in sports and physical therapy, in that some pressure mapping devices can analyze people only in static settings but not dynamic settings. In other words, some pressure mapping devices can analyze people when they are standing but not when they are walking or running. The locomotion device(s) 112 disclosed herein can address these issues by using an FTIR-based scanning technique to provide improved spatial resolution for measuring health parameters integrated into a dynamic setting. The locomotion device 112 can be an exercise or physical therapy device, such as a treadmill machine 130, an elliptical machine 702, a leg press 704, a stair stepping machine 706, and/or a row machine 708, incorporating the transparent medium 104 for performing the health parameter characterization techniques.

FIGS. 4A and 4B depict an example health parameter detection device 102 having an exercise machine form factor 402 that integrates the health parameter detection device 102 into the locomotion device 112. The locomotion device 112 can include a treadmill machine 130. The treadmill machine 130 can trap light (e.g., from the plurality of LEDs 108) inside a flexible running belt 132 formed of the transparent medium 104, which the subject moves on (e.g., stands, walks, and/or runs on). Multiple cameras 404 can record footprints causing pressure and/or FTIR events on the transparent medium 104, and then a processing unit can transform the footprints to pressure maps.

In some examples, the treadmill machine 130 can include one or more motors 406 connected to one or more rollers for generating rotational motion for the one or more rollers 408. The treadmill machine 130 can also include a frame 410 to which the other components are fastened/mounted. The flexible running belt 132 can wrap around the one or more rollers 408. Furthermore, the treadmill machine 130 can include an incline mechanism for adjusting a height dimension at one side of the treadmill machine 130, and a belt fastening mechanism for fastening the flexible running belt 132 in place. Furthermore, the treadmill machine 130 can include the one or more cameras 106, such as a plurality or array 414 of cameras in an interior portion of the treadmill machine 130 directed up towards the transparent medium 104. Additionally, the treadmill machine 130 can include one or more light sources, such as the plurality of LEDs 108 for providing light into the flexible running belt 132.

As depicted in FIGS. 4A and 4B, the treadmill machine 130 can omit any running deck by using a flexible running belt 132 made of flexible yet strong material that can hold both the frictional and normal forces while a user is running on it. The light from the plurality of LEDs 108 can be trapped inside the flexible running belt 132 on which the subjects moves on, and multiple cameras 106 can record the footprints and/or scattered light caused by the footprints. The processing unit can be used to transform the footprints into pressure maps, perfusion maps, and so forth.

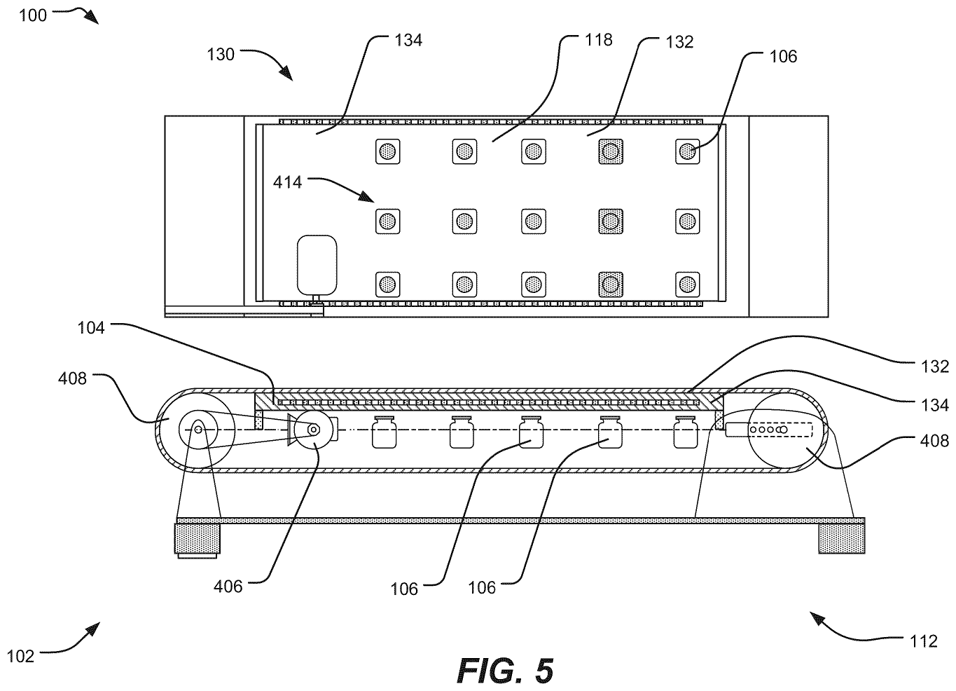

FIG. 5 depicts an example treadmill machine 130 which includes the features of the treadmill machine 130 discussed above regarding FIGS. 4A and 4B, and also includes a rigid deck 134. For instance, the flexible running belt 132 in this design can be a flexible thin rubber-like or a fabric-like material that provides the friction forces but omits providing the normal forces. The rigid deck 134 can be disposed at least partially below the flexible running belt 132 and can provide the normal forces, which can support the subject while the subject is standing, walking, and/or running on the flexible running belt 132. Light from the plurality of LEDs 108 can be trapped inside the rigid deck 134. Multiple cameras 106 can be used to record footprints and/or scattered light caused by the footprints. Then the processing unit can transform the footprints into pressure maps, perfusion maps, and so forth.

In this configuration, the rigid deck 134 can be formed of a rigid material and can provide the normal forces. The rigid deck 134 can also operate as the FTIR domain (e.g., the transparent medium 104) in which the light from the plurality of LEDs 108 is trapped. Multiple cameras 106 (e.g., forming a row of cameras or an array 414 of cameras 106), one camera 106, and/or a camera-mirror module can be positioned under the running deck to record the footprints and/or scattered light caused by the footprints. An internal or external processing unit can then transform the images or videos into pressure maps, perfusion maps, and so forth. The internal or external processing unit can also perform post-processing of the data to create the analytics outputs of the data for visual display via one or more graphical user interfaces.

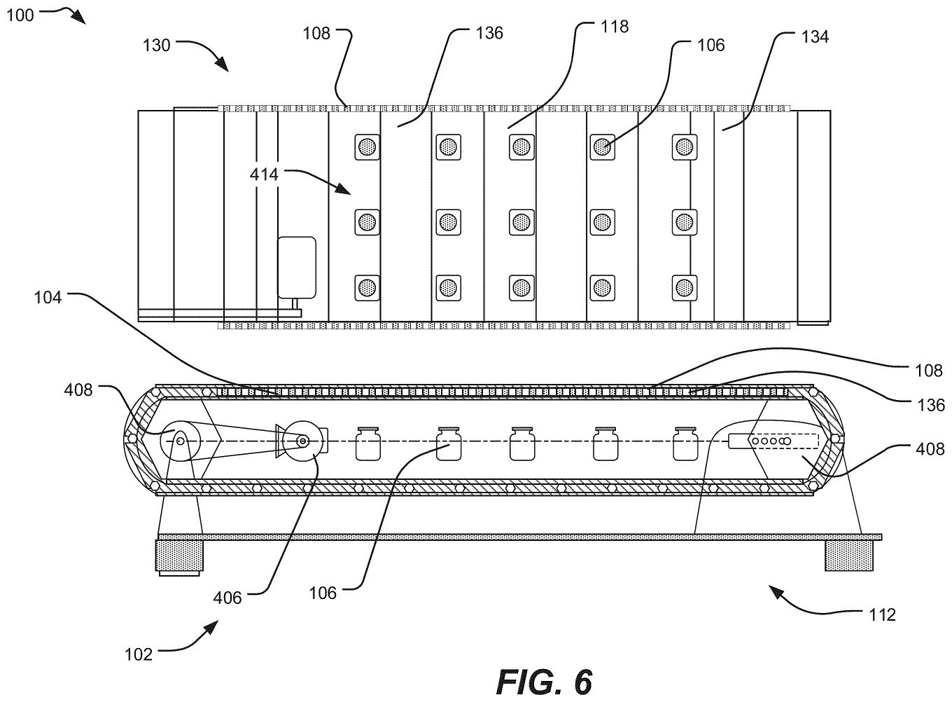

FIG. 6 depicts an example treadmill machine 130 which can include any of the features discussed above regarding FIGS. 4A-5. Furthermore, as depicted in FIG. 6, the treadmill machine 130 can include a plurality of rigid planks 136, or rigid substantially rectangular bars, which can be formed of the transparent medium 104.

In this configuration, the flexible running belt 132 and/or the rigid deck 134 can be replaced with several rigid rectangular bars or rigid planks 136 that provide both the normal and frictional forces. The light from the plurality of LEDs 108 can be trapped inside the bars and one or multiple cameras 106 or camera-mirror modules can be used to record the pressure map (e.g., footprints). The light can be trapped inside the bars in two different ways. First, the plurality of LEDs 108 can be attached to the plurality of rigid planks 136 and can move with the plurality of rigid planks 136. Second, the plurality of LEDs 108 can be stationary and/or fixed onto the structure of the treadmill machine 130 (e.g., a frame of the treadmill machine 130), and the plurality of rigid planks 136 can move past the plurality of LEDs 108 while the plurality of LEDs 108 are emitting light, such that the light becomes trapped inside the plurality of rigid planks 136. Additionally, the internal or external computing modules can be used to process the data.

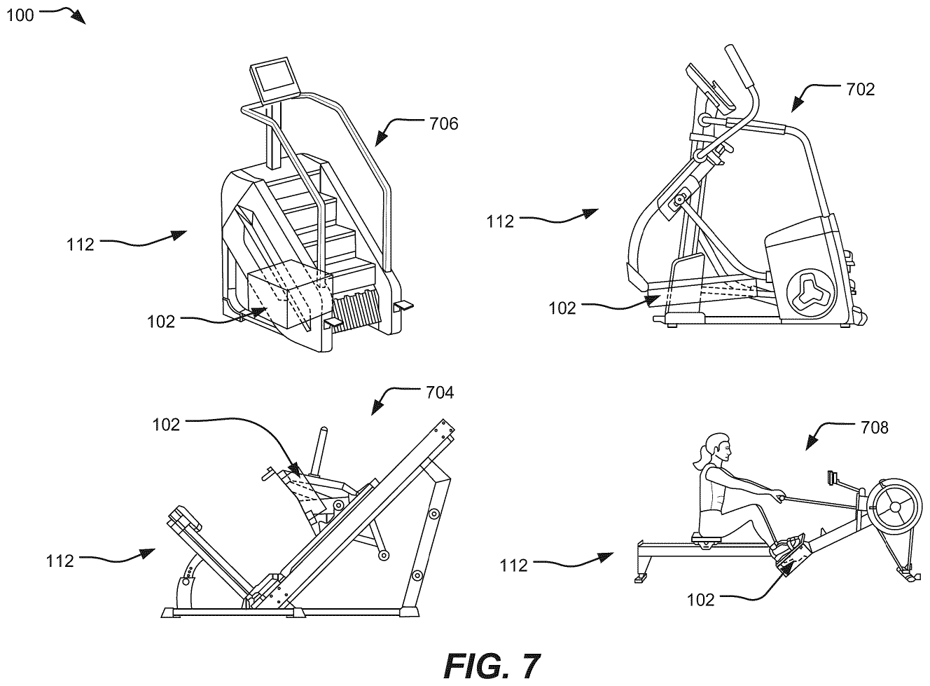

FIG. 7 depicts an example system 100 including different form factor implementations of the locomotion device 112. The system 100 depicted in FIG. 7 can form at least a portion of the system 100 depicted in FIG. 1.

For instance, the health parameter detection device 102 can be integrated into several different designs to form different types of pressure mapping devices such as fitness machines and/or physical therapy machines. The locomotion device 112 can include a stair stepping machine 706 with the health parameter detection device 102 at least partly integrated into one or more steps of the stair stepping machine 706. The locomotion device 112 can include an elliptical machine 702 with the health parameter detection device 102 at least partly integrated into the standing platforms portion of the elliptical machine 702. The locomotion device 112 can include a row machine 708 with the health parameter detection device 102 at least partly integrated into the footrest portion of the row machine 708. The locomotion device 112 can include a leg press machine 704 with the health parameter detection device 102 at least partly integrated into a foot press portion of the leg press machine 704. Various other types of exercise equipment, physical therapy equipment, medical diagnosis equipment, and/or health parameter monitoring equipment may integrate the health parameter detection device 102.

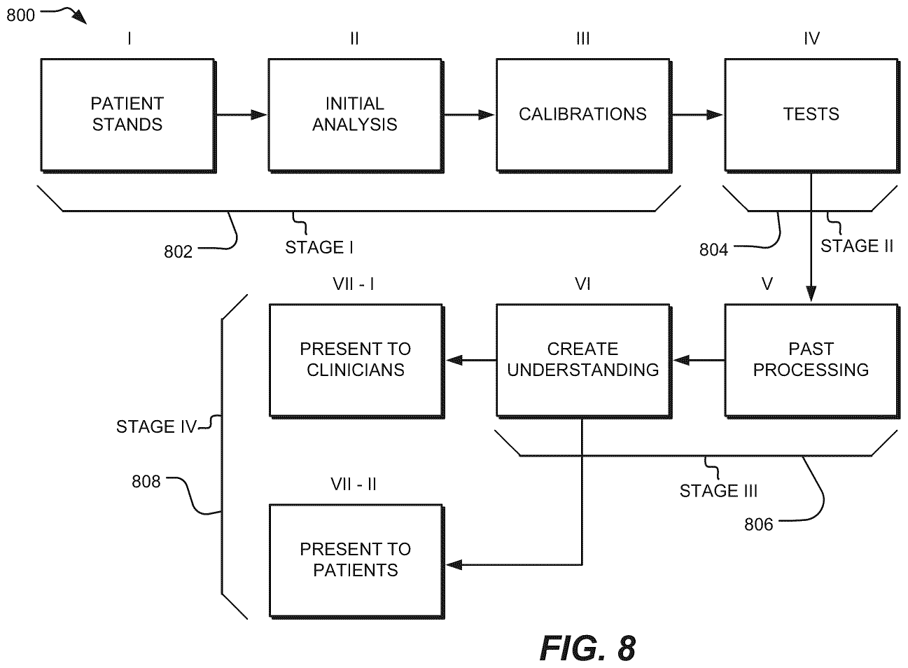

FIG. 8 depicts an example method 800 for using a health parameter detection device 102 to determine a health parameter of a subject. The method 800 can be performed by any of the systems 100 depicted in FIGS. 1-7.

In some examples, the method 800 can include at least four different stages during an examination of a patient or subject, and the different stages can each include different parts. This method 800 can be performed using one or more computing devices, such as a clinician computing device, a cloud server device, one or more mobile devices, and so forth. The one or more computing devices can locally execute applications and/or can connect to each other through one or more network connections. The one or more computing devices can include a non-transitory computer-readable storage device storing instructions which, when executed by one or more processors, performs one or more of the operations discussed herein. For instance, the computing device(s) can perform the health parameter characterization procedure by performing various data collection, analysis, and other input/output operations, as discussed below.

In some instances, the health parameter characterization procedure can include a first stage for initialization and calibration (e.g., an initialization/calibration stage 802). For example, the initialization/calibration stage 802 can include taring the force sensors and/or taking pretesting images of the glass (e.g., the transparent medium 104) to calibrate the images for possible dirt and/or scratches of the transparent medium 104, or light noise from the room, or possible light noise from the plurality of LEDs 108.

The initialization/calibration stage 802 can include one or more subjects standing on the sample surface 105 of the health parameter detection device 102 for a predetermined amount of time (e.g., 2 to 3 minutes) to determine viscoelastic properties of the tissue contacting the sample surface 105. A predetermined set of different wavelengths of light can be emitted for calibration purposes. Particular wavelengths of light can be emitted at a beginning and/or an end of the calibration process, which can then be recorded. For example, white light can be emitted at the beginning to analyze the optical properties of the foot. The light emitted from the sample surface 105 during the calibration process can be from both the FTIR effect and/or from shining a light directly on the foot which reflects the light to the light sensor. As such, the method 800 can include determining optical properties of the skin by first recording an image of the skin with or without white light, and then performing an FTIR-based analysis on the skin with a calibration factor to account for the optical properties of the skin. As such, the optical properties of the tissue contacting the sample surface 105 can be calibrated.

In some examples, the first stage (e.g., the initialization/calibration stage 802) can include calibrating the health parameter detection device 102 for the skin color of the subjects, the viscoelastic mechanical properties of the foot, and/or a roughness factor measurement. In this way, the method 800 can account for the roughness of the foot in pressure measurements by at least one of generating an FTIR-based surface topography and/or inclusion of other types of 3D scanners, such as a projection method. Furthermore, a force measurement and/or center of gravity measurement can be determined as part of the first stage (e.g., the initialization/calibration stage), which can further be used to normalize and/or calibrate the test data analysis equations. Also, before the subject stands on the glass, a calibration process can be run to identify characteristics of the health parameter detection device 102 (e.g., the transparent medium 104), such as impacts from scratches, previous footprints, dirt, and so forth.

In some instances, the method 800 includes a second stage, which can be a testing and/or experiments stage 804. For instance, the subject can stand on the transparent medium 104 in a normal standing position for a first predefined period of time. Then the user can stand in a vestibular inhibition pose on the sample surface for a second predefined period of time. A balance distribution difference between the two poses can then be calculated. Additionally testing data can be collected in various other poses (e.g., standing on one leg, crouching, squatting, jumping, etc.), which can also be used to determine the balance distribution, the pressure maps, the perfusion map, and so forth.

In some examples, the method 800 includes a third stage which can be a post processing stage 806. For instance, using one or more computing devices, the health parameter detection device 102 can normalize the testing data for optical properties of foot based on the calibration initialization stage. The health parameter detection device 102 can normalize the testing data for a roughness factor of the foot based on the calibration initialization stage. The health parameter detection device 102 can normalize the testing data for anthropometry based on the calibration initialization stage. The health parameter detection device 102 can normalize the testing data based on patient demographic data. In some examples, one or more machine-learning algorithms can use a training process and/or a validation process (e.g., with or without human assistance) to perform the post processing steps of the method 800 and/or calculate the pressure distribution.

In some instances, the method 800 includes a fourth stage which can be a data presentation stage 808. There can be several different ways to present the post-processed testing data to clinicians and patients/subjects. For instance, the data can be presented via a graphical user interface on a display as one or more graphs, lists, spreadsheets, charts, icons, and so forth to represent the post-processed testing data.

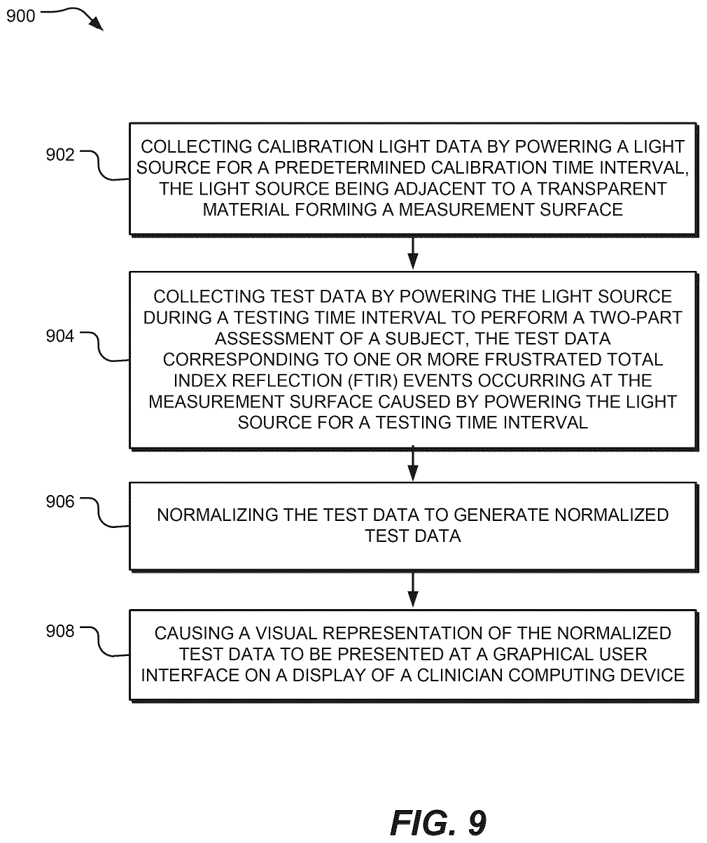

FIG. 9 depicts an example method 900 for health parameter detection that can be performed with the health parameter detection device 102. The method 900 can be performed by any of the systems depicted in FIGS. 1-7.

In some examples, at operation 902, the method 900 can collect calibration light data by powering a light source for a predetermined calibration time interval, the light source being adjacent to a transparent material forming a measurement surface. At operation 904, the method 900 can collect test data by powering the light source during a testing time interval to perform a two-part assessment of a subject, the test data corresponding to one or more frustrated total internal reflection (FTIR) events occurring at the measurement surface caused by powering the light source for a testing time interval. At operation 906, the method 900 can normalize the test data to generate normalized test data. At operation 908, the method 900 can cause a visual representation of the normalized test data to be presented at a graphical user interface on a display of a clinician computing device.

It is to be understood that the specific order or hierarchy of steps in the method(s) depicted throughout this disclosure are instances of example approaches and can be rearranged while remaining within the disclosed subject matter. For instance, any of the operations depicted throughout this disclosure may be omitted, repeated, performed in parallel, performed in a different order, and/or combined with any other of the operations depicted throughout this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A health parameter detection device comprising:
a body;
a platform formed of a transparent material, the platform including a measurement surface forming a first surface of the body, the measurement surface is operable to contact a target area of a user;
one or more light sources disposed adjacent to the transparent material and operable to transmit light into the transparent material;
a mirror disposed at an interior of the body and forming a first angle with the measurement surface; and
a camera disposed at least partially in the body, such that the camera is operable to receive a scattered light caused by a frustrated total internal of reflection (FTIR) event occurring at the measurement surface and reflecting from the mirror.

2. The device of claim 1, further comprising:
one or more wheels extending from a second surface of the body.

3. The device of claim 2, further comprising:
a handle formed into a third surface of the body.

4. The device of claim 3, further comprising:
a mounting frame disposed between the platform and the mirror, substantially parallel to the platform; and
one or more force sensors, disposed between the platform and the mounting frame, operable to detect a force applied to the measurement surface.

5. The device of claim 1,
wherein,
the one or more light sources includes a row of light emitting diodes (LEDs) disposed along a side of the transparent material.

6. The device of claim 1,
wherein,
the mirror includes a first mirror; and
the device further includes:
a second mirror in the interior of the body forming a third angle with the measurement surface such that the second mirror receives light reflected from the first mirror; and
a third mirror in the interior of the body forming a fourth angle with the measurement surface such that the third mirror reflects light, reflected from the second mirror and received at the third mirror, towards the camera.

7. The device of claim 6,
wherein,
the third mirror is a curved mirror.

8. The device of claim 6, further comprising:
a lens positioned between the third mirror and the camera.

9. The device of claim 8,
wherein,
the one or more light sources include a plurality of different frequency LEDs; and
the scattered light is caused by a plurality of FTIR events resulting from powering the plurality of different frequency LEDs, one frequency at a time, such that the health parameter detection device constructs a nano scale or micro scale 3D topology from the plurality of FTIR events.

10. A health parameter detection system comprising:
a platform formed of a transparent material, the platform including a measurement surface operable to contact a target area of a user;
a light source operable to transmit light into the transparent material;
one or more cameras directed at the transparent material such that the one or more cameras is operable to receive a scattered light caused by a frustrated total internal reflection (FTIR) event occurring at the measurement surface because of contact with the target area; and
one or more motors operable to move the measurement surface.

11. The system of claim 10, further comprising:
a computer-readable memory device storing instructions that, when executed by one or more processor, cause the health parameter detection system to:
collect calibration light data by powering the light source for a predetermined calibration time interval, the light source including a calibration set of light emitting diodes (LEDs),
collect test data by performing a two-part assessment of a subject,
normalize the test data using the calibration light data, anthropometric data, or patient demographic, to generate normalized test data, and
cause a visual representation of the normalized test data to be presented at a graphical user interface on a display of a clinician computing device.

12. The system of claim 11,
wherein,
the instructions, when executed by the one or more processor, cause the health parameter detection system to generate a skin surface calibration profile indicating one or more optical properties of a bottom surface of a foot.

13. The system of claim 10,
wherein,
the one or more cameras includes a plurality of cameras forming an array directed at an underside of the platform.

14. The system of claim 10,
wherein,
the platform includes at least one of:
a flexible running belt including the transparent material for receiving the light from the light source, or
a rigid deck around which the flexible running belt wraps, the rigid deck including the transparent material for receiving the light from the light source.

15. The system of claim 10,
wherein,
the platform includes a plurality of parallel, rigid planks extending from a first side of the platform to a second side of the platform.

16. The system of claim 10,
wherein,
the platform forms part of at least one of:
a treadmill machine,
an elliptical machine,
a rowing machine,
a stair stepping machine, or
a leg press machine.

17. A method of health parameter detection, the method comprising:
collecting calibration light data by powering a light source for a predetermined calibration time interval, the light source being adjacent to a transparent material forming a measurement surface;
collecting test data by powering the light source during a testing time interval to perform a two-part assessment of a subject, the test data corresponding to one or more frustrated total internal reflection (FTIR) events occurring at the measurement surface caused by powering the light source for a testing time interval;
normalizing the test data to generate normalized test data; and
causing a visual representation of the normalized test data to be presented at a graphical user interface on a display of a clinician computing device.

18. The method of claim 17,
wherein,
the light source includes a calibration set of light emitting diodes (LEDs) including LEDs of different frequencies corresponding to a contact surface of a calibration subject.

19. The method of claim 17,
wherein,
the two-part assessment includes:
generating first position data corresponding to a position of the subject, and
generating second position data corresponding to the position with vestibular inhibition.

20. The method of claim 19,
wherein,
the test data includes a balance distribution generated by calculating a difference between the first position data and the second position data.
